(19) United States
(12) Patent Application Publication
(10) Pub. No.: US 2013/0171255 A1
(43) Pub. Date: Jul. 4, 2013

(54) LAXATIVE

(71) Applicant: Detlef Schierstedt, St. Augustin (DE)

(72) Inventor: Detlef Schierstedt, St. Augustin (DE)

(73) Assignee: KRUEWEL MUESELBACH GmbH, Eitorf (DE)

(21) Appl. No.: 13/817,499

(22) PCT Filed: Sep. 1, 2011
(86) PCT No.: PCT/EP2011/065131

§ 371 Date: Mar. 11, 2013

(30) Foreign Application Priority Data
Sep. 7, 2010 (DE) 20 2010 012 256.2

(51) Int. Cl. A61K 9/20; A61K 9/14; A61K 9/00

(52) U.S. Cl. CPC A61K 9/20; A61K 9/00; A61K 9/14
USPC 424/474; 514/723; 424/490

(57) ABSTRACT

The invention relates to a laxative with improved compliance, containing polyethylene glycol (PEG). A medicinal product according to the invention is suitable for treating chronic obstipations and is characterized in that it comprises two components, A and B, which are spatially separated from one another, component A comprising a polyethylene glycol (PEG) and component B comprising at least one electrolyte.

The present invention relates to a laxative containing polyethylene glycol (PEG) and having an improved compliance. A medicinal product according to the invention is suitable for treating chronic constipation.

“Constipation” means the delayed defecation of dry and hard stool. It can be attributed to either decelerated intestinal passage, or impaired defecation reflex. Causes of the delayed intestinal passage may be dietetic factors, changes of the intestinal wall, endocrine disorders, and functional and organic disorders of the nervous system. Also, medicaments, for example, sedatives, psychoactive drugs or opioids, may have a constipating effect. An impaired defecation reflex is found in diseases of the anal canal, in case of a loss of the rectal dilation reflex, or weakness in the muscles used to apply abdominal pressure.

If a low roughage diet is the cause of a delayed intestinal passage, a change of the living and eating habits is mostly sufficient. This frequently results in a temporary use of laxatives, which accelerate defecation. Most laxatives act by increasing the intraluminal volume and thus trigger peristaltic waves by increasing the internal pressure in the intestine. Basically, three groups of laxatives that have such an effect can be distinguished:
swelling agents, which swell within the intestine, absorbing water;
osmotic laxatives, which draw water into the intestine and/or retain it therein by osmotic pressure;
laxatives that inhibit the absorption of Na⁺ ions and thus of water from the intestinal lumen and/or enhance water secretion into the lumen.

Further, lubricants, which are supposed to facilitate defecation by a lubricant effect, and pharmaceuticals that trigger the defecation reflex are employed.

The temporary intake of laxatives very rarely causes severe disorders. In contrast, administering them chronically leads to disorders of electrolyte metabolism, which in turn may increase the constipation. Just for a prolonged indication, for example, in opioid-related constipation, this effect occurs if the electrolytes are not sufficiently substituted.

Naturally occurring or synthetically prepared swellable, non-digestible polysaccharides, such as linseed or Indian plantago (psyllium), which swell inside the intestine, are suitable as mild laxatives. They must be ingested together with sufficient water in order to avoid gelatinization of the intestinal contents.

The widely known castor oil inhibits the absorption of sodium ions and water by blocking the sodium-potassium-dependent ATPase. The laxative effect is reliable. However, since it is only reluctantly used, it is more suitable for therapy of acute constipation.

Today, laxatives are employed very frequently. Many OTC products are heavily advertised in the media. The corresponding agents, for example, Dulcolax® Balance, are based on the effect of polyethylene glycol. However, they are merely suitable for application over a short period of less than four weeks. Prolonged use may lead to loss of electrolyte, which cannot be compensated by these agents.

Polyethylene glycol, a polymeric powder, has an osmotic effect. The powder binds the water with which it is swallowed, transporting it into the large intestine. There, the osmotic pressure is locally increased, and water is released into the intestinal lumen. Polyethylene glycol is neither absorbed nor metabolized (Mutschler Arzneimittelwirkungen: Lehrbuch der Pharmakologie and Toxikologie, by E. Mutschler et al., 8th Edition, Stuttgart: Wissenschaftliche Verlagsgesellschaft mbH, 2001, pp. 647-652).

For prolonged use, the addition of electrolytes, for example, sodium chloride or potassium chloride, is required. The effectiveness of corresponding polyethylene glycol electrolyte solutions was shown in clinical studies (A. Attar et al.: Comparison of a low dose polyethylene glycol electrolyte solution with lactulose for treatment of chronic constipation, Gut 1999; 44: 226-230).

The presence of the electrolytes in a polyethylene glycol solution leads to an unpleasant taste of this solution (salty taste). Patients who have to ingest PEG electrolyte solutions over a period of several months develop an aversion to the solution because of the intensive salty taste. This results in a poor compliance with respect to regular ingestion. However, just in the case of chronic constipation, a regular ingestion over an extended period of time is necessary.

Thus, the object of the present invention is to provide a product for treating chronic constipation with improved compliance.

The core of the invention is the fact that the compliance can be significantly improved if the polyethylene glycol and electrolyte are taken in separately.

In a first embodiment, the object of the present invention is achieved by a medicinal product for treating chronic constipation, characterized by comprising two physically separated components A and B, wherein component A comprises a polyethylene glycol (PEG) and component B comprises at least one electrolyte.

The polyethylene glycol preferably has a molecular weight within a range of from 2000 g/mol to 6000 g/mol, especially within a range of from 3000 g/mol to 4000 g/mol, especially of 3350 g/mol. The corresponding polyethylene glycols have a particularly good osmotic effect in the intestine, which is mainly responsible for the activity as a laxative. For example, a particular amount of PEG 2000 (polyethylene glycol having a molecular weight of 2000 g/mol) causes approximately twice as high an osmotic pressure as the same amount of PEG 4000 (polyethylene glycol having a molecular weight of 4000 g/mol).

If the molecular weight of the PEG is too low, the PEG has a bitter taste. This results in a reduced compliance. If the molecular weight of the PEG is too high, its osmotic effect is too low. In this case, a sufficient laxative effect is no longer achieved.

To achieve a sufficient laxative effect, the daily dose is usually within a range of from 7 g to 40 g, preferably from 13 g to 26 g, of PEG per day. With an ingested amount of less than 7 g of PEG per day, a sufficient laxative effect cannot be ensured. If the amount of ingested PEG is significantly higher than 40 g per day, diarrheas may result. The ingestion of 13 g to 26 g of PEG per day has come to be a particularly preferred dose for adults. However, this value depends on individual conditions, such as the body weight or the cause of constipation.

In one embodiment of the present invention, the medicinal product according to the invention includes 13.125 g of PEG in component A as a unit dose. This corresponds to a standard daily dose for an adult. When the demand is higher, up to three unit doses per day can be taken.

Further, component A may additionally include one or more flavoring agents, which additionally improve the compliance. “Flavoring agents” within the meaning of the present application means flavors, such as orange flavor, or acidulants, such as citric acid. Further, component A may also comprise sweetening agents, such as saccharin-sodium, sugar and/or sodium cyclamate, etc.

The contained PEG itself is almost tasteless. The addition of flavoring agent and/or sweetening agent according to the invention results in a pleasant taste of the medicinal product. Intensity of flavor and sweetness can be adjusted by varying these substances. Because of the pleasant taste, aversion to the medicinal product according to the invention does not develop even after prolonged use, resulting in a significantly improved compliance even over an extended period of use.

This is important, in particular, in opioid-related constipation. The corresponding patients often have to use pain killers, for example, analgesics, over an extended period of time, leading to chronic constipation. In this case, if a patient does not or not regularly use a laxative, this may result in a deterioration in general condition. Also, balance of the electrolytes is of critical importance just in this context, because impaired electrolyte balance can also lead to deterioration of the general condition.

In one embodiment according to the invention, component A is in the form of granules, especially granules for oral solution, or as a ready-made solution.

In addition to component A, which comprises the polyethylene glycol, the medicinal product according to the invention further comprises at least one electrolyte. The latter is physically separated as component B.

For example, NaCl and/or KCl, and optionally additionally sodium hydrogencarbonate, may be used as the electrolyte. Further, Mg salts and/or Ca salts, for example, as a citrate, and/or additional inulin may also optionally be further added.

Component B, which comprises the electrolyte, may be in the form, for example, of coated granules or powder, as a tablet, film tablet, capsule, and/or coated tablet. In particular, component B is in the form of a film tablet.

For the preparation of a tablet, capsule, film tablet and/or coated tablet according to the invention, the usual excipients can be used.

In another embodiment, the object of the present invention is achieved by a kit comprising at least two separate containers, a container 1 containing component A of the medicinal product, and another container 2 containing component B of the medicinal product.

According to the invention, container 1 may be a sachet. In particular, sachets of coated aluminum are employed. Such a sachet is suitable for ingesting either the granules containing the polyethylene glycol, or the ready-made drinking solution. The drinking solution can also be filled into bottles for one and/or several applications.

Component B, which contains the electrolyte, is preferably in a solid form. Thus, the corresponding container 2 may be a sachet and/or a deep drawn sheet. Thus, the electrolyte tablets according to the invention may be contained in blister strips.

According to the invention, container 1 and container 2 may also be detachably connected with each other. However, it must be possible to separate the two containers in such a way that the respective containers 1 and 2 remain intact. This can be achieved, for example, by means of a perforation. During the separation, components A and B should not become mixed.

The separate ingestion of components A and B ensures a significant improvement of compliance. The latter is also maintained over a longer period of time. Also, supply of the necessary electrolytes is ensured.

### EXAMPLES

### Example 1

### Component A: Granules:

| | |
|---|---|
| Macrogol ® 3350 | 13.125 g |
| Saccharin sodium | 0.005 g |
| Citric acid | 0.1 g |
| Orange flavor | 0.25 g |

### Component B: Tablet:

| | |
|---|---|
| NaCl | 0.351 g |
| KCl | 0.47 g |
| Avicel ® | 0.3 g |
| Actisol ® | 0.1 g |
| Mg stearate | 0.005 g |

### Example 2

### Component A: Solution in Aluminum Sachet:

| | |
|---|---|
| Macrogol ® 3350 | 13.125 g |
| H₂O | 13.2 g |
| Saccharin sodium | 0.005 g |
| Sodium cyclamate | 0.05 g |
| Orange flavor | 0.131 g |
| Citric acid | 0.131 g |

### Component B: Tablet:

| | |
|---|---|
| NaCl | 0.351 g |
| KCl | 0.047 g |
| Sodium hydrogencarbonate | 0.179 g |
| Dicalcium phosphate × 2H₂O | 0.3 g |
| Actisol ® | 0.1 g |
| Mg stearate | 0.009 g |

1-10. (canceled)

11. A medicinal product for treating chronic constipation, comprising two physically separated components A and B for separate ingestion, wherein component A comprises a polyethylene glycol (PEG) in the form of granules for oral solution, or as an aqueous solution and component B comprises at least one electrolyte as coated granules, powder, tablet, film tablet, capsule, or coated tablet.

12. The medicinal product according to claim 11, characterized in that said polyethylene glycol has a molecular weight within a range of from 2000 g/mol to 6000 g/mol.

13. The medicinal product according to claim 11, characterized in that component A further comprises one or more flavoring agents.

14. The medicinal product according to claim 13, characterized in that said flavoring agent comprises flavors and/or acidulants and/or sweetening agents.

15. The medicinal product according to claim 11, characterized in that component A is in the form of granules for oral solution.

16. The medicinal product according to claim 11, characterized by containing as electrolytes NaCl and/or KCl.

17. A kit comprising at least two separate containers, a first container containing component A of the medicinal product according to claim 11, and a second container containing component B of the medicinal product according to claim 11.

18. The kit according to claim 17, characterized in that said first container is a sachet, or a bottle.

19. The kit according to claim 17, characterized in that the second container is a sachet or a deep-drawn sheet.

20. The medicinal product according to claim 12, characterized in that said polyethylene glycol has a molecular weight within the range of from 3000 g/mol to 4000 g/mol.

21. The medicinal product according to claim 16, characterized by further containing sodium hydrogencarbonate.

22. The medicinal product according to claim 16, characterized by further containing Mg salts and/or Ca salts.

23. The medicinal product according to claim 22, wherein said Mg salts and/or Ca salts are in the form of citrate.

24. The medicinal product according to claim 16, characterized by further containing inulin.
